(19) United States
(12) Patent Application Publication
(10) Pub. No.: US 2002/0015729 A1
(43) Pub. Date: Feb. 7, 2002

(54) COLONIC DELIVERY OF WEAK ACID DRUGS

(76) Inventor: PETER JAMES WATTS, NOTTINGHAM (GB)

Correspondence Address: PATREA L. PABST, HOLLAND & KNIGHT LLP, 1201 WEST PEACHTREE STREET, ONE ATLANTIC CENTER, SUITE 2800, ATLANTIC, GA, 30309-3400, US

(21) Appl. No.: 09/269,903

(22) PCT Filed: Oct. 6, 1997
(86) PCT No.: PCT/GB97/02726

(30) Foreign Application Priority Data
Oct. 4, 1996 (GB) 9620709.7

(51) Int. Cl. A61K 9/16; A61K 9/50; A61K 9/58; A61K 9/26; A61K 9/28; A61K 9/36

(52) U.S. Cl. 424/462; 424/490; 424/480; 424/469; 424/474; 424/493

(57) ABSTRACT

There is provided a controlled release formulation including an inner core comprising, or coated with, a drug, which drug possesses (a) a free acid group which can be converted into an alkali metal salt and (b) a pKa in the rnage 2.0 to 9.0, which inner core is subsequently coated with a rate-controlling membrane that determines drug release, wherein the drug is present as a salt that displays higher solubility at pH 4.5 to 8.0 than the corresponding compound containing a free acid group.

This invention relates to novel controlled-release formulations of drugs with pKa values of between 2.0 and 9.0.

Drugs that have weak basic functions and/or weak acid functions (i.e. those with pKa values of between 2.0 and 9.0) often have a low and/or variable solubility at pH values normally experienced in the colon (i.e. between 4.5 and 8.0). Consequently, if a drug is delivered to the colon for e.g. local action, the dissolution of the drug from the tablet, pellet or capsule formulation can be extremely variable, resulting in unsatisfactory controlled release profiles.

Ridogrel ((E)-5-[[[3-pyridinyl[3-(trifluoromethyl)phenyl]methylene]amino]oxy]pentanoic acid; Janssen Pharmaceutica, Belgium; see U.S. Pat. No. 4,963,573) is an example of a drug in which such problems have been found to occur. Ridogrel is a development compound which has been indicated for use in the treatment of inter alia inflammatory bowel diseases including Crohn's disease and ulcerative colitis. The drug may be administered orally in simple pharmaceutical formulations. However, it is anticipated that, if the drug could be delivered to the colonic region of the gastrointestinal tract in a slow release (rate-controlled) fashion, advantages would result. For example, delivery to the colon is likely to concentrate the drug at the required site of action and therefore prevent unwanted absorption of the drug into the systemic circulation from the small intestine. Further, the controlled release nature of such a formulation is likely to provide good distribution of the drug to the various regions of the large intestine.

General methods for the site specific delivery of drugs to the large bowel have been described in the prior art, including the applicant's pending international patent application WO 95/35100, which discloses the coating of starch capsules with polymers that degrade or dissolve under the conditions found within the different regions of the gastrointestinal tract. In this prior art document, a preferred system was disclosed as comprising a starch capsule coated with a mixture of methacrylate polymers. These polymers only dissolve at pH values above 4.5, thereby allowing a formulation to remain intact in the stomach. Upon entry into the small intestine, the coating on the capsule begins to dissolve. By adjustment of the thickness of the coating of such formulations, it is possible for the capsule to reach the terminal ileum or ascending colon before releasing its contents.

Another granted patent (EP 513 035) describes how a similar effect can be achieved using polymers that are degraded specifically in the colonic environment due to the unique reducing conditions therein. Polymers based upon disulphide bonds have been shown to be effective both in vitro and in vivo.

Alternatively, compositions can be delivered to the colon using other known colon targeting systems. Some examples, which are not exhaustive, are as follows:

The Time Clock Release System™ (Pozzi et al, APV Course on Pulsatile Drug Delivery, Konigswinter, May 20, 1992) is a tablet system where a tablet core containing the active drug is coated with a layer of pharmaceutical excipients. The excipients hydrate causing the surface layer to burst at a set time. The Pulsincap™ system is an oral pulsatile delivery system which may be configured to release its drug content at a predetermined time or place within the gastrointestinal tract. The device essentially consists of an impermeable capsule body which contains the drug, sealed at the neck orifice with a hydrogel plug. A normal gelatin cap is then placed onto the body of the device. After ingestion, the gelatin cap dissolves allowing the plug to hydrate. At a predetermined and controlled time, the swollen plug is ejected from the body of the device, thereby releasing the capsule contents and enabling the drug to be released (Wilding et al., Pharm. Res. 9, 654, 1992 and Binns et al., 3rd Eur. Symp. Control. Drug Del., Abstract Book, 1994, p124).

Another system which may be used is the time controlled explosion system, as described in U.S. Pat. No. 4,871,549.

The problem to be solved in the case of the drug ridogrel and similar molecules (for example those which are weakly ionisable in nature, in particular those which are useful in the treatment of inflammation of the intestines and especially thromboxane synthase A₂ inhibitors and thromboxane A₂/prostaglandin endoperoxide receptor antagonists such as those disclosed in U.S. Pat. No. 4,963,573) is one of (a) achieving a controlled release formulation that will provide good distribution throughout the colon in order to optimise treatment of affected sites, and (b) for such release to be constant (i.e. as near to zero order as possible) and predictable (i.e. reproducible) over an extended period of time.

Controlled release formulations of drugs which target the colon in particular may also be useful for the systemic delivery of therapeutic agents as “once daily” products.

A variety of formulation principles have been disclosed in the prior art for the controlled release of drugs that are weak acids or weak bases. However, it has been found that, in order for a formulation to be distributed evenly at the target site, a multiparticulate pellet formulation is preferred. Pellets may be formed by a number of different processes, all well known in the art, including extrusion and spheronisation, as well as the coating of the drug material onto preformed sugar spheres (also known as non-pariels). The drug can be coated onto non-pariels using techniques which are familiar to those skilled in the art. A controlled release layer may then be coated on top of the drug layer so as to provide a diffusional barrier. Unfortunately, with drugs such as ridogrel, we have found that a simple diffusional barrier does not provide a satisfactory product. This is because ridogrel has weakly basic functions and a carboxylic acid function and the solubility of the drug in the colonic pH range (4.5 to 8.0) is therefore low, resulting in extremely variable dissolution of the drug at such pH values. Thus, a simple formulation, wherein ridogrel is coated onto non-pariel beads, and then overcoated with a rate-controlling membrane, does not result in a formulation possessing a satisfactory release profile.

However, we have found, surprisingly, that it is possible to achieve a satisfactory formulation comprising drugs such as ridogrel by choosing, instead of the drug itself, an appropriate salt (e.g. alkali metal salt) that has pH independent solubility characteristics. The salt of the drug should be at least 10 times more soluble than the free acid form of the drug and, more preferably, greater than 100 times more soluble, as measured in deionized water at the relevant pH range (i.e. 4.5 to 8.0) at 37° C. By “more soluble” we mean that the salt is more soluble over the entire pH range of 4.5 to 8.0. It is then found, surprisingly, that the coated pellet system gives an almost pH independent release profile under in vitro conditions as tested in the USP type 2 dissolution apparatus (The United States Pharmacopoeia, USP 23, 1994, page 1791-1793), for example as described hereinafter.

The pellet system comprising drug may be coated with a coating material (a rate controlling membrane). The nature and thickness of this coating material may be altered (for example as described hereinafter) to provide a controlled release formulation which will, for example, release the drug over a period of up to 5 hours or over a longer period of up to 12 hours. The present invention thus provides a controlled release formulation comprising an inner core containing, or coated with, a drug and subsequently coated with a rate-controlling membrane that determines drug release, of a drug that contains a weak acid function with a pKa in the range 2.0 to 9.0 (e.g. 3.0 to 9.0) that can be converted into an alkali metal salt wherein the drug is present as a salt that displays higher solubility at pH 4.5 to 8.0 (e.g. 5.0 to 7.0) than the corresponding compound containing a free acid group.

Thus, according to a first aspect of the invention, there is provided a controlled release formulation including an inner core comprising, or coated with, a drug, which drug possesses (a) a free acid group which can be converted into an alkali metal salt and (b) a pKa in the range 2.0 to 9.0 (e.g. 3.0 to 9.0), which inner core is subsequently coated with a rate-controlling membrane that determines drug release, wherein the drug is present as a salt that displays higher solubility at pH 4.5 to 8.0 (e.g. 5.0 to 7.0) than the corresponding compound containing a free acid group (referred to hereinafter as “the compositions according to the invention”).

Drugs which may be employed in the compositions according to the invention include those which have a rapidly changing solubility in the pH range 4.5 to 8.0 (i.e. the pH range found in the colon under normal conditions and/or those conditions reported to exist in acute conditions such as ulcerative colitis). Drugs which may be employed include ridogrel, other thromboxane synthase A₂ inhibitors and thromboxane A₂/prostaglandin endoperoxide receptor antagonists (such as those disclosed in U.S. Pat. No. 4,963,573), and sodium cromoglycate. Particularly preferred drugs include ridogrel.

Suitable salts of the weak acid drugs include ammonium salts and particularly alkali metal salts such as, but not limited to, sodium and potassium salts. Such salts may be prepared in accordance with techniques which are well known to those skilled in the art, including, in the case of alkali metal salts, dissolving the drug in a solution of the relevant hydroxide. For example, an excess of drug may be suspended in the hydroxide solution and stirred for 24 hours. The suspended material may then be removed by filtration and centrifugation and the salt recovered from the filtrate by removal of the water (e.g. using a vacuum oven or by lyophilisation).

The salts may also be prepared as part of a preparation process for the coating of the inner cores. In this case, drug is dissolved in, for example, an appropriate hydroxide solution at a suitable concentration (e.g. 1M) and the pH is adjusted to about 8 by adding acid, such as 0.1M HCl. The salt solution may then be added to a solution of a binder (such as povidone) and the pH adjusted to about 8 (again). This mixture may then be coated onto the inner cores using, for example, a spray coating apparatus. The pellets may, if necessary, be overcoated with a thin layer of plasticised HPMC, which may act as a “primer”, in order to obtain a better coating The inner cores may then be overcoated with the controlled release coating layer (rate-controlling membrane), which may, for example, consist of Eudragit® RS30D, triethyl citrate and talc, and subsequently dried. The pellets may then be filled into capsules to be coated for delivery to the colon, or compressed into tablets which are then coated.

The inner core may comprise drug salt. Drug salt may be incorporated into the inner core during the manufacture of the latter, for example by extrusion/spheronisation.

Inner cores which may be employed in the compositions according to the invention include sugar spheres (non-pariels). Suitable sizes of inner cores which may be employed in the compositions according to the invention are in the range 0.3 to 5 mm.

In general, the preferred controlled release coating materials which may be employed in the rate-controlling membrane of the compositions according to the invention include those which form a water-insoluble but water-permeable layer and from which release of drug is by diffusion through the layer. By “water-insoluble” we mean “sparingly soluble” as defined in the British Pharmacopoeia (1988). By “water-permeable” we mean that at least 10% of water, held continuously in contact with the layer, will penetrate the layer within two hours (the degree of permeation may be measured in accordance with techniques which are well known to those skilled in the art). The coating polymer may be inherently water-permeable or become water-permeable through the incorporation of other additives such as plasticisers or pore forming agents. Suitable coating polymers include methacrylate copolymers, ethylcellulose, etc. Preferred coating materials are the permeable, water insoluble grades of pharmaceutical polymethacrylates (Eudragit® RL100, Eudragit RS100/RS30D, Eudragit NE30D, Rohm Pharma, Darmstadt, Germany) and ethylcellulose. Eudragit RL100 and RS100 contain quaternary ammonium groups which may interact with ionised weakly acidic drugs and hence the most preferred coating materials are ethylcellulose and Eudragit NE30D. Ethylcellulose may be applied as a solution in an organic solvent or as a proprietary water-based latex preparation (e.g. Aquacoat®, FMC, Philadelphia, USA or Surelease®, Colorcon, West Point, USA).

The thickness of the rate-controlling membrane required for use in the compositions according to the invention will depend on the permeability of the polymer to the drug in question and the duration of release required from the coated formulation. However, the amount employed will typically be in the range 2% w/w to 25% w/w of the formulation, or will be an amount to produce a thickness in the range 80 μm to 300 μm.

The compositions according to the invention may be adapted to deliver therapeutic agent to the colonic region of the gastrointestinal tract, especially the proximal colon. Preferably, a means is provided to prevent release of drug until the formulation reaches the colonic region.

By “colonic region of the gastrointestinal tract” we mean the terminal ileum and the colon.

The compositions according to the invention, may thus be filled into the various known delivery systems intended for targeting the colonic region, including those described above, and including the coated capsules described above. Alternatively, the compositions according to the invention may be further coated with an enteric layer that slowly dissolves within the small intestine to allow exposure of the rate-controlling membrane to the liquid in the terminal ileum and/or the colon for subsequent release. In a similar fashion to the coated starch capsules disclosed in international patent application WO 95/35100, the coating may be an enteric polymer that dissolves in the small intestine or a polymeric or polysaccharide material that is not degraded until it meets the specific conditions found in the colon. Such degradation may be through direct chemical effect, e.g. the degradation of disulphide bonds under reducing conditions, or the degradation of polysaccharide materials under the effects of the microflora found within the colon.

Preferred coating materials for targeting to the colon, which may be used in capsules, tablets or pellets including the compositions according to the invention, are those which dissolve at pH of 4.5 or above. In this way, the coatings only begin to dissolve once they have left the stomach and have entered the small intestine. A thick layer of coating is thus preferably provided which will dissolve in about 2 to 5 hours, thereby allowing the capsule underneath to break-up only when it has reached the terminal ileum and/or the colon. Such a coating can be made from a variety of polymers such as cellulose acetate trimellitate (CAT), hydroxypropylmethyl cellulose phthalate (HPMCP), polyvinyl acetate phthalate (PVAP), cellulose acetate phthalate (CAP) and shellac, as described by Healy in his article “Enteric Coatings and Delayed Release”, Chapter 7 in Drug Delivery to the Gastrointestinal Tract, eds. Hardy et al, Ellis Horwood, Chichester, 1989. For coatings of the polymers, a thickness of 150 to 300 μm is suitable

Especially preferred materials are methylmethacrylates or copolymers of methacrylic acid and methylmethacrylate. Such materials are available as Eudragit® enteric polymers (Rohm Pharma, Darmstadt, Germany; see above). These are copolymers of methacrylic acid and methylmethacrylate.

Preferred compositions are based on Eudragit L100 and Eudragit S100. Eudragit L100 dissolves at pH 6 and upwards and comprises 48.3% methacrylic acid units per g of dry substance; Eudragit S100 dissolves at pH 7 and upwards and comprises 29.2% methacrylic acid units per g of dry substance. Preferred coating compositions are based on Eudragit L100 and Eudragit S100 in the range 100 parts L100:0 parts S100 to 20 parts L100:80 parts S100. The most preferable range is 70 parts L100:30 parts S100 to 80 parts L100:20 parts S100. As the pH at which the coating begins to dissolve increases, the thickness necessary to achieve colon specific delivery decreases. For formulations where the ratio of Eudragit L100:S100 is high, a coat thickness of the order 150-200 μm is preferable. This is equivalent to 70-110 mg of coating for a size 0 capsule. For coatings where the ratio Eudragit L100:S100 is low, a coat thickness of the order 80 to 120 μm is preferable, which is equivalent to 30 to 60 mg coating for a size 0 capsule.

The colonic region has a large population of microbial anaerobic organisms providing reducing conditions. Thus, the coating may suitably comprise a material which is redox-sensitive. Such coatings may comprise azopolymers which may, for example, consist of a random copolymer of styrene and hydroxyethyl methacrylate, cross-linked with divinylazobenzene synthesised by free radical polymerisation (the azopolymer being broken down enzymatically and specifically in the colon), or disulphide polymers (see PCT/BE91/00006 and Van den Mooter, Int. J. Pharm. 87, 37 (1992)).

Other materials which may be used to provide release in the colon include amylose. For example, a coating composition can be prepared by mixing amylose-butan-1-ol complex (glassy amylose) with Ethocel® aqueous dispersion (Milojevic et al., J. Control. Rel., 38, 75 (1996)), or a coating formulation comprising an inner coating of glassy amylose and an outer coating of cellulose or acrylic polymer material (Allwood et al., GB9025373.3), calcium pectinate (Rubenstein et al., Pharm. Res., 10, 258, (1993)), pectin, a polysaccharide which is totally degraded by colonic bacterial enzymes (Ashford et al., Br. Pharn. Conference, 1992 Abstract 13), chondroitin sulphate (Rubenstein et al., Pharm. Res. 9, 276, 1992) and resistant starches (Allwood et al., PCT WO89/11269, 1989), dextran hydrogels (Hovgaard and Brφndsted, 3rd Eur. Symp. Control. Drug Del., Abstract Book, 1994, 87), modified guar gum, such as borax modified guar gum (Rubenstein and Gliko-Kabir, S. T. P. Pharma Sciences 5, 41 (1995)), p-cyclodextrin (Sie ke et al., Eur. J. Pharm. Biopharm. 40 (suppl.), 335 (1994)), saccharide containing polymers, by which we include a polymeric construct comprising a synthetic oligosaccharide-containing biopolymer, including methacrylic polymers covalently coupled to oligosaccharides such as cellobiose, lactulose, raffinose, and stachyose, or saccharide-containing natural polymers including modified mucopolysaccharides such as cross-linked chondroitin sulfate and metal pectin salts, for example calcium pectate (Sintov and Rubenstein; PCT/US91/03014); methacrylate-galactomannan (Lehmann and Dreher, Proc. Int Symp. Control. Rel. Bioact. Mater. 18, 331 (1991)), pH-sensitive hydrogels (Kopecek et al., J. Control. Rel. 19, 121 (1992)) and resistant starches, e.g. glassy amylose, that are not broken down by the enzymes in the upper gastrointestinal tract but are degraded by enzymes in the colon.

It will be well understood by those skilled in the art that further excipients may be employed in the compositions according to the invention For example, further excipients which may be employed include diluents such as microcrystalline cellulose (e.g. Avicel®, FMC), lactose, dicalcium phosphate and starch(es); disintegrants such as microcrystalline cellulose, starch(es) and cross-linked carboxymethylcellulose; lubricants such as magnesium stearate and stearic acid; granulating agents such as povidone; and release modifiers such as hydroxypropyl methylcellulose and hydroxypropyl cellulose. Suitable quantities of such excipients will depend upon the identity of the active ingredient(s) and particular dosing form which is used.

Appropriate quantities of drug salts which may be employed in the compositions according to the invention will depend upon the agent which is used. However, it will be clear to the skilled person that doses of drug salts can be readily determined non-inventively. Suitable doses for selected drugs in the present invention (e.g. ridogrel) are in the range 1 to 200 mg, preferably 2 to 100 mg and more preferable, 5 to 50 mg.

Compositions according to the invention have been found to have the advantage that they provide an improved release profile in respect of drugs which have a rapidly changing solubility, and therefore an extremely variable dissolution, in the colonic pH range (4.5 to 8.0).

Thus, according to a further aspect of the invention there is provided a method of improving the release profile of a drug with a rapidly changing solubility in the pH range 4.5 to 8.0 which method comprises administering a composition according to the invention to a patient, preferably a human patient.

In view of the advantageous properties of the compositions according to the invention, they are useful in the treatment of conditions such as ulcerative colitis, Crohn's disease, irritable bowel syndrome and/or inflammatory bowel disease, when adapted for delivery to the colonic region.

According to a further aspect of the invention there is provided a method of treatment of ulcerative colitis, Crohn's disease, irritable bowel syndrome and/or inflammatory bowel disease which method comprises administering a composition according to the invention to the colonic region of a patient, preferably a human patient.

### BRIEF DESCRIPTION OF THE FIGURES

FIG. 1 shows the release of ridogrel at pH 6 and pH 7 from 0.61 to 0.7 mm pellets coated with 3.7% Eudagrit RS (USP method 2; 37° C.).

FIG. 2 shows the dissolution of (a) ridogrel and (b) sodium ridogrel at pH 5, 6 and 7.

FIG. 3 shows the release of ridogrel (as the sodium salt) at pH 5, 6 and 7 from 0.6 to 0.71 mm pellets coated with 19% w/w Eudagrit RS (USP method 2; 37° C.).

FIG. 4 shows the release of ridogrel (as the sodium salt) from 1 to 1.18 mm pellets with three levels of Aquacoat coating (USP method 2; 37° C.).

FIG. 5 shows the release of ridogrel (as the sodium salt) at pH 5, 6 and 7 from 1 to 1.18 mm pellets containing 14% Aquacoat coating (USP method 2; 37° C.).

FIG. 6 shows the dissolution performance of starch capsules containing inner cores comprising sodium ridogrel.

FIG. 7 shows the plasma profiles of three colon targeted formulations as determined in a human clinical trial, pharmacoscinitigraphy study.

The invention is illustrated, but in no way limited, by the following examples.

### EXAMPLE 1 (Comparative Example)

Preparing Ridogrel Pellets Coated with Polymethacrylate (Eudragit RS)

A solution of 20 g of ridogrel (Janssen Pharmaceutica; Belgium) and 2 g of povidone (Kollidon 30) in 250 mL of ethanol was prepared. This solution was spray-coated onto 400 g of sugar spheres (600-710 μm, NP Pharma, France) using an Aeromatic STREA-1 coater. The pellets were assayed for ridogrel content by a spectrophotometric method. To prepare the coating solution of sustained release polymer, 35 g of talc was first dispersed in 250 mL of water and 9 g of triethyl citrate was added. 150 mL of Eudragit RS30D (Rohm Pharma) was then added to the talc dispersion. 280 g of the ridogrel-coated pellets were then coated with the Eudragit solution in the STREA-1 using an inlet temperature of 50° C. 100 mL of solution was applied to the pellets. The pellets were then dried overnight at 40° C. and assayed for ridogrel content using a spectrophotometric method (UV).

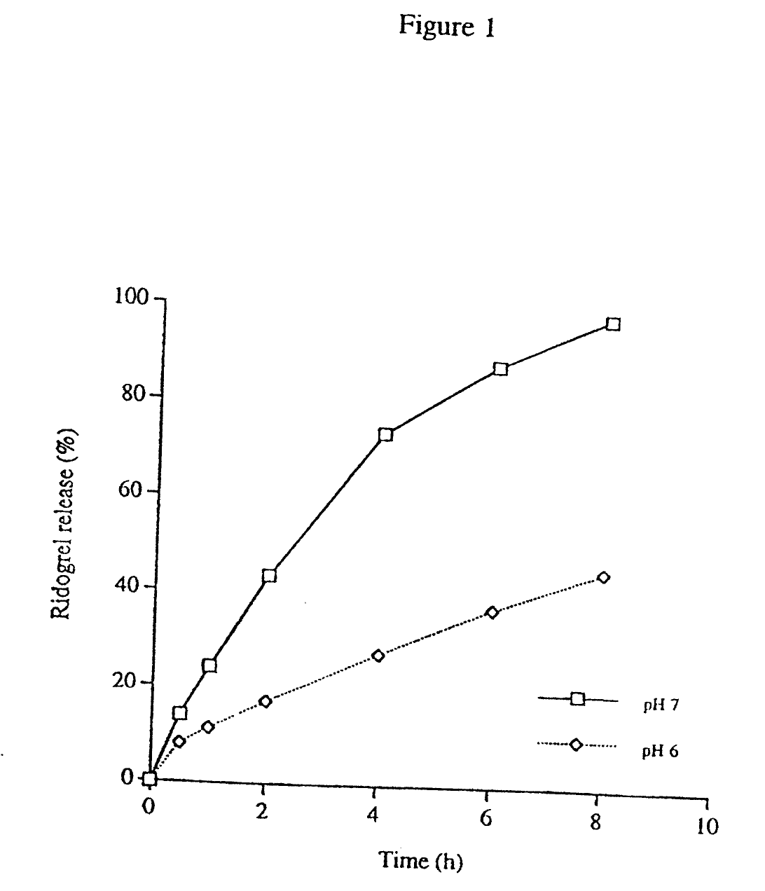

The dissolution performance of the pellets was measured using the BP/USP method 2 (USP23, 1994, page 1791-1793; paddles, 50 rpm) with 900 mL of either pH 6 or pH 7 phosphate buffer as the test medium. In FIG. 1, the dissolution performance of the pellets is shown. Compared to the performance of the pellets at pH 7, there was a substantial reduction in the rate of drug release at pH 6. For example, after 4 hours, approximately 24% of the ridogrel had been released at pH 6, compared to 74% at pH 7.

### EXAMPLE 2

Solubility of Ridogrel and Sodium Ridogrel

In accordance with the invention, sodium ridogrel was prepared as follows:

i) 0.1 g of sodium hydroxide was dissolved in 20 mL of water;

ii) 1.5 g of ridogrel was added to the sodium hydroxide solution to form a suspension;

iii) the ridogrel suspension was placed into a sonic bath for 10 minutes;

iv) the suspension was passed through a 0.45 μm membrane filter, the filtrate was collected, diluted by adding 20 mL of water, and lyophilised overnight; and

v) the lyophilised sodium ridogrel was gently milled in a mortar to produce a fine powder.

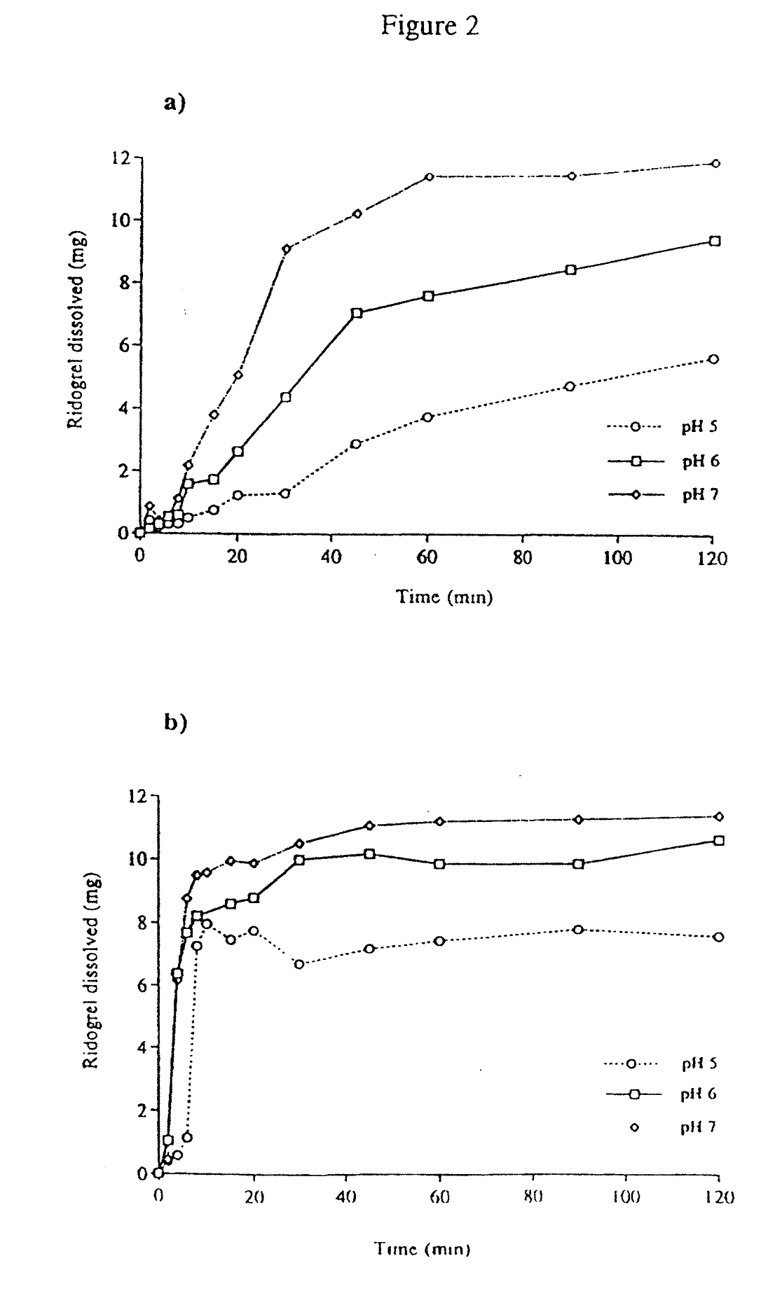

Into each of three size 2 hard gelatin capsules was weighed 10 mg of ridogrel. Into another three capsules was weighed 10 mg of the sodium ridogrel lyophilisate. The dissolution of ridogrel and sodium ridogrel into 900 mL of phosphate buffer at pH 5, 6 and 7 was tested (USP apparatus 2, 100 rpm). The dissolution rate of ridogrel (as the parent acid) increased as the pH was raised (FIG. 2a). In contrast, the rate of dissolution of sodium ridogrel was largely independent of pH (FIG. 2b).

Therefore there was a significant reduction in the rate of dissolution of ridogrel as the pH was reduced from 7 to 5, the pH range likely to be encountered in the large intestine However, in this pH range, the sodium salt of ridogrel had a greatly improved dissolution rate.

### EXAMPLE 3

Preparation of Pellets Coated with Sodium Ridogrel and Eudragit

Pellets were prepared containing the sodium salt of ridogrel. 20 g of ridogrel was dissolved in approximately 60 ml of 1M sodium hydroxide solution. The solution of sodium ridogrel was adjusted down to pH 8 by adding 0.1M hydrochloric acid and made up to 100 mL with water. 40 g of povidone (Kollidon 30; BASF) was dissolved in 200 mL of water. The povidone solution was added to the ridogrel solution and a precipitate was formed, which was dissolved by adding sodium hydroxide to adjust the solution to pH 8. The povidone/sodium ridogrel solution was applied to 1 kg of sugar spheres (0.6-0.71 mm) using the Aeromatic STREA-1 coater After coating, the pellets were relatively tacky which could have been due to the hygroscopic nature of the povidone and/or the sodium ridogrel. To remove this tackiness, the pellets were overcoated with a thin layer of HPMC: The HPMC solution was prepared by dissolving 30 g of Methocel® E5 in 600 mL of water and adding 3 g of PEG400 as a plasticiser. The pellets were assayed for ridogrel content.

450 mL of Eudragit coating solution was prepared as follows: 150 mL of Eudragit RS30D, 9 g of triethyl citrate, 35 g of talc, 250 mL of water. The solution was applied 400 g of sodium ridogrel/povidone/HPMC pellets. The coated pellets were dried overnight at 40 °C. The pellets were assayed for ridogrel content.

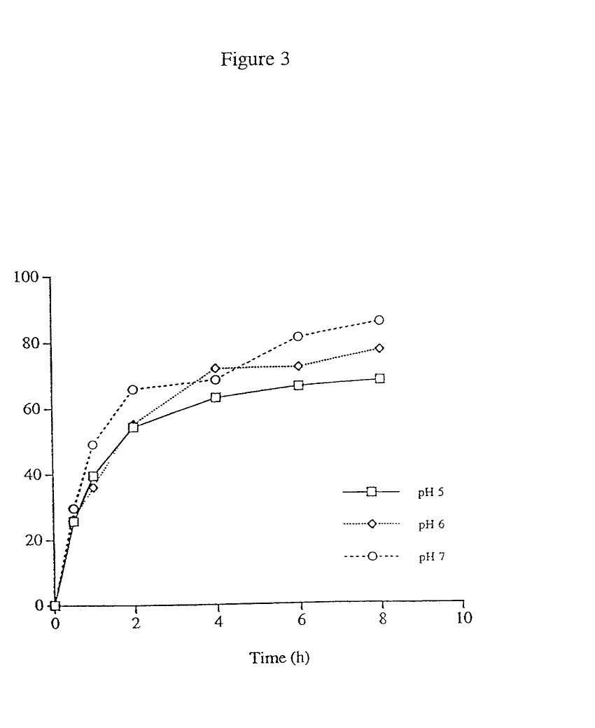

The dissolution performance of the pellets at pH 5, 6 and 7 is shown in FIG. 3. There was a small reduction in the rate of drug release as the pH was decreased. This demonstrated that the rate of release of ridogrel as the sodium salt was largely independent of pH, which was in marked contrast to pellets containing ridogrel as the parent acid (see FIG. 1).

### EXAMPLE 4

Preparation of Pellets Coated with Sodium Ridogrel and Ethylcellulose

Pellets were prepared with an ethylcellulose outer layer. A water-based ethylcellulose preparation, Aquacoat® (FMC, Philadelphia), was used in order to eliminate the use of organic solvents in the coating process. Pellets were prepared as follows:

20 g of ridogrel was weighed into a beaker and dissolved in 56 mL of 1M sodium hydroxide solution. 40 g of povidone (Kollidon K30) was weighed into a large beaker and dissolved in 500 mL of water. The ridogrel solution was added to the povidone solution. The pH change resulted in precipitation of ridogrel. Sodium hydroxide solution was added to dissolve the ridogrel. The pH of the solution was adjusted to pH 8 using 0.1M hydrochloric acid and made up to 600 mL with water. 1 kg of sugar spheres (1.00-1.18 mm diameter) were coated with the sodium ridogrel/povidone solution using the Aeromatic STREA-1 coater (inlet temperature was 55° C.)

An overcoat of HPMC was applied to the sodium ridogrel/povidone layer. The HPMC solution was prepared by dispersing 20 g of HPMC (Methocel® E5) in 200 mL of hot water. The dispersion was cooled in ice (whilst being stirred) and 2 g of PEG400 was added as a plasticiser. The solution was made up to 400 mL with water The HPMC solution was applied using the STREA-1 at an inlet temperature of 55° C. The completed pellets were left to dry overnight at room temperature. The Aquacoat mixture was prepared by stirring together 300 mL of Aquacoat and 21.6 g of dibutyl sebacate for 1 hour, followed by the addition of 300 mL of water. 500 g of sodium ridogrel/povidone/HPMC pellets were transferred to the Aeromatic and coated with the Aquacoat mixture (coating temperature 40° C.). Pellet samples (20 g) were collected at intermediate points in the coating run, after the application of approximately 300 mL and 450 mL of the coating solution. After coating, the pellet samples were spread into trays and dried overnight at 60° C.

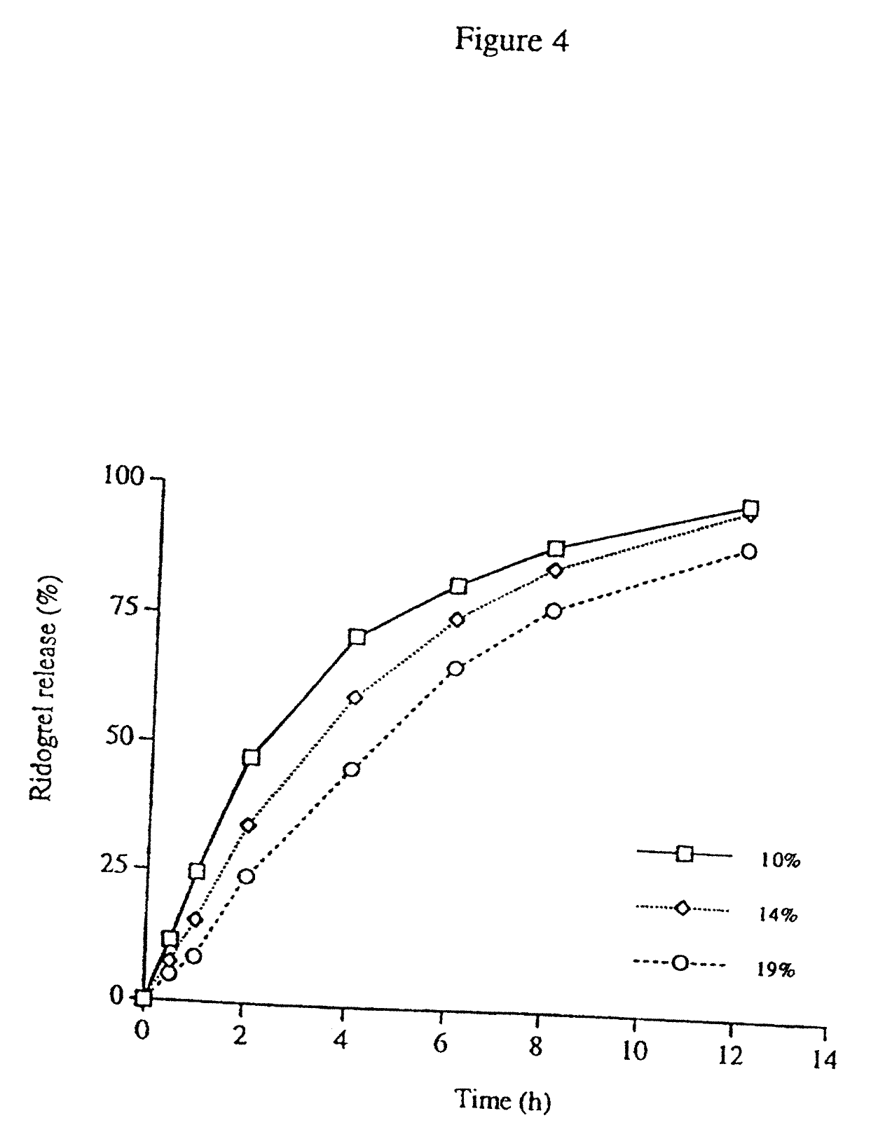

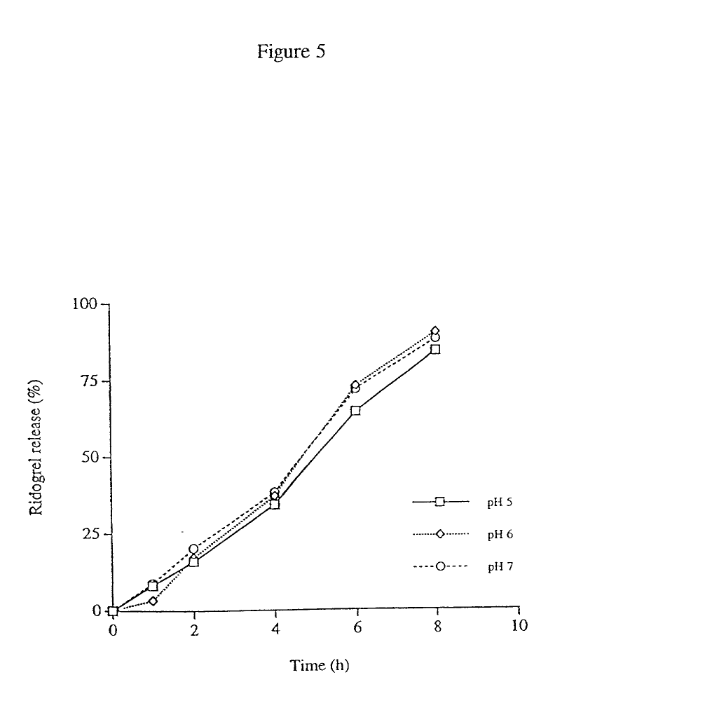

The dissolution performance of the pellets at pH 7 is shown in FIG. 4. The dissolution performance of the pellets containing 14% coating at pH 5, 6 and 7 is shown in FIG. 5. Drug release was independent of pH. The release of drug from these samples was complete. This was in contrast to the Eudragit-coated pellets where drug release was incomplete. This was probably due to an interaction between negatively-charged ridogrel ions and positively charged quaternary ammonium groups within Eudragit RS. Hence ethylcellulose is a preferred polymer for use in preparing ridogrel controlled release pellets.

### EXAMPLE 5

Preparation of Formulations for Testing in Human Clinical Trial, Phase 1

Pellets were prepared with an ethylcellulose outer layer as the rate controlling membrane. A water based ethylcellulose preparation, Aquacoat® (FMC, Philadelphia) was used. 10 g of ridogrel was weighed into a beaker and dissolved in 28 mL of 1M sodium hydroxide solution and made up to 100 mL with water. 20 g of povidone (Kollidon K 30) was weighed into a large beaker and dissolved in 200 mL of water. The ridogrel solution was added to the povidone solution. 1M sodium hydroxide solution was added to dissolve the precipitated ridogrel and the pH was adjusted to 8 with 0.1M hydrochloric acid.

500 g of sugar pellets (1-1.18 mm in diameter) were coated with the sodium ridogrel/povidone solution using the aromatic STREA-1 coater (inlet temperature 55° C.). An overcoat of HPMC was applied to the sodium ridogrel/povidone layer. The HPMC solution was prepared by dispersing 20 g of HPMC (Methocel E5) in 200 mL hot water. The dispersion was cooled in ice (whilst being stirred) and 1 g of PEG 400 was added as a plasticiser and the volume made up to 400 mL with water. The HPMC solution was applied using the STREA-1 at an inlet temperature of 55° C. The completed pellets were left to dry overnight at room temperature. 30 g of pellets were removed (“immediate release pellets”; A).

The Aquacoat mixture was prepared by stirring together 300 mL of Aquacoat and 21.6 g of dibutyl sebacate for 1 hour, followed by the addition of 300 mL of water. About 500 g of sodium ridogrel/povidone/HPMC pellets were transferred to the Aeromatic STREA-1 coater and coated with Aquacoat mixture (coating temperature 45° C.). Pellet samples of 35 g were collected after the application of 450 mL (“8 hour release pellets”; B) and after the application of 600 mL (“12 hour release pellets”; C) of Aquacoat. After coating the pellet samples were spread into trays and dried overnight at 60° C.

The three different pellet samples were filed into starch capsules (Capill) with approximately 425 mg in each capsule. The capsules were coated with a Eudragit solution consisting of Eudragit S100/Eudragit L100 1:3, dibutyl sebacate, talc, isopropanol and water in the Aeromatic STREA-1 coater. The coating conditions used were drying temperature 25° C., fan speed 6, atomisation pressure 1 bar and application rate 1.5-4.0 mL/minute. The weight gain per capsule was 78 mg.

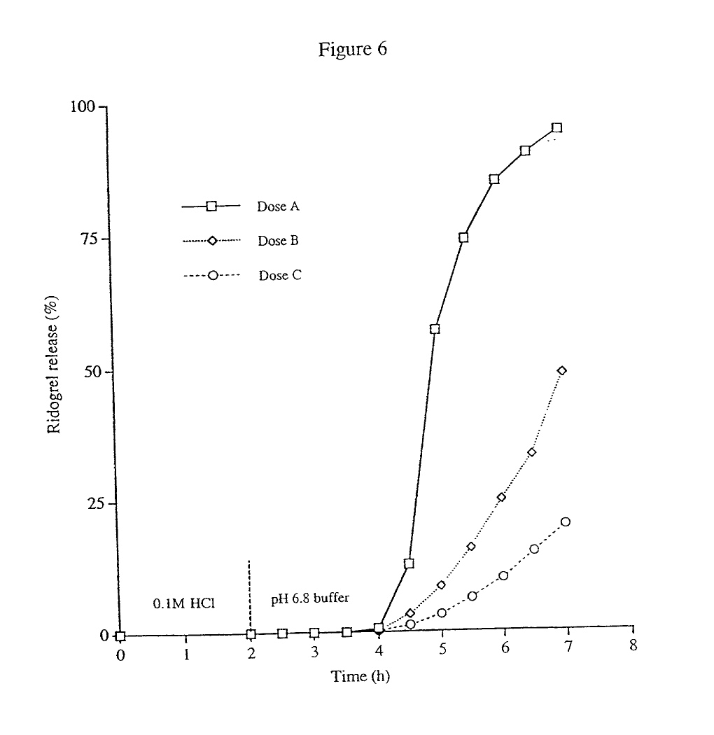

The dissolution performance of the capsules at 37° C. for 2 hours in 0.1M HCl, followed by phosphate buffer, pH 6.8 in a Vankel 6010 dissolution apparatus (baskets rotated at 50 rpm) is shown in FIG. 6. (Values are the mean of the two determinations.) The difference in rate of dissolution between the 3 different pellet samples is clearly seen.

### EXAMPLE 6

Phase 1 Human Clinical Trial, Pharmacoscintigraphy Study

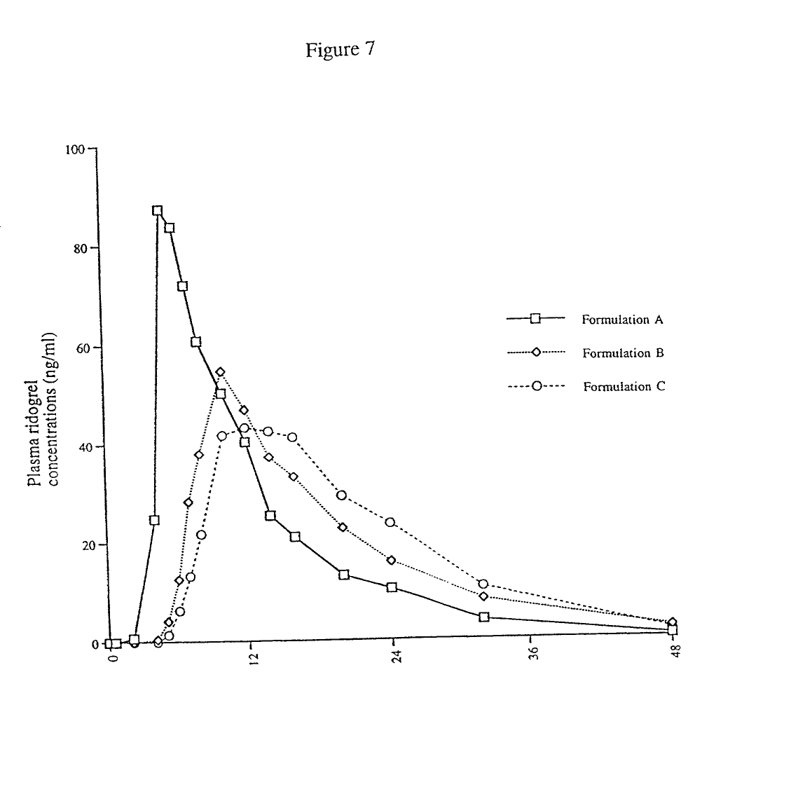

The clinical trial was a four way crossover study in 8 healthy male volunteers, aged 18-35 years. Three of the doses administered were the colon targeted capsule formulations described in Example 5. These formulations were radiolabelled with a gamma emitting isotope (indium-111). The fourth formulation was a conventional immediate release tablet, and was not radiolabelled. On each study day, blood samples were collected for ridogrel analysis. Plasma samples were analysed by Janssen Pharmaceutica. Of the capsules dosed, 21 disintegrated at the ileocaecal junction or in the colon and two in the lower small intestine. The plasma ridogrel analysis showed that for all three colon targeted formulations, peak plasma concentrations occurred much later than with the conventional tablet (7.5 h, 12.5-13 h as compared with 0.9 h). The maximum plasma ridogrel concentrations were much lower for the colon targeted formulations than of the conventional tablet, and plasma concentrations were sustained for a longer period. Furthermore the maximum plasma concentration for the immediate release, colon targeted formulation was higher than for the sustained release formulations. The plasma profiles for the colon targeted formulations are shown in FIG. 7. (Values are the mean for all volunteers, omitting those where the dose was retained in the stomach. For Formulation A, n=6; Formulation B, n=7; Formulation C, n=8.)

1. A controlled release composition including pellets, each comprising an inner core, which core comprises, or is coated with, a drug, which drug possesses (a) a free acid group which can be converted into an alkali metal salt and (b) a pKa in the range 2.0 to 9.0, which inner core is subsequently coated with a rate-controlling membrane that determines drug release, wherein the drug is present as a salt that displays higher solubility at pH 4.5. to 8.0 than the corresponding compound containing a free acid group, and wherein the composition is adapted to prevent release of drug until the composition reaches the terminal ileum or the colon.

2. A composition as claimed in claim 1 wherein the drug is a thromboxane synthase A₂ inhibitor or a thromboxane A₂/prostaglandin endoperoxide receptor antagonist.

3. A composition as claimed in claim 2 wherein the drug is ridogrel.

4. A composition as claimed in any one of claims 1 to 3, wherein the rate-controlling membrane comprises a material which forms a water-insoluble but water-permeable layer and from which release of drug is by diffusion through the layer.

5. A composition as claimed in claim 4, wherein the rate-controlling membrane is formulated from a methacrylate copolymer or ethylcellulose.

6. A composition as claimed in claim 5, wherein the rate-controlling membrane is formulated from ethylcellulose or Eudragit NE30D.

7. A composition as claimed in claim 6 where the rate-controlling membrane is ethylcellulose.

8. A composition as claimed in any one of the preceding claims, wherein the inner core is a sugar sphere.

9. A composition as claimed in any one of the preceding claims, wherein the salt is at least 10 times more soluble than the free acid form of the drug at pH 4.5 to 8.0 at 37° C.

10. A composition as claimed in any claim 9, wherein the salt is at least 100 times more soluble than the free acid form of the drug.

11. A composition as claimed in any one of the preceding claims, wherein the salt is an alkali metal salt.

12. A composition as claimed in claim 11, wherein the alkali metal is sodium or potassium.

13. A composition as claimed in any one of the preceding claims wherein the pellets are administered in a starch capsule coated with a combination of polymethacrylates that is designed to disintegrate and release the pellets in the terminal ileum or in the colon.

14. A composition as claimed in any one of the preceding claims wherein the drug is used for the treatment of ulcerative colitis, Crohn's disease, irritable bowel syndrome, inflammatory bowel disease.

15. A process for the preparation of a composition according to any one of the preceding claims which comprises making a salt of the drug and coating said salt onto the inner cores.

16. A process as claimed in claim 15, wherein the salt is prepared as part of a preparation process for the coating of the inner cores.

17. A method of improving the release profile of a drug with a rapidly changing solubility in the pH range 4.5 to 8.0, which comprises administering a composition according any one of claims 1 to 14 to a patient, preferably a human patient.

18. The use of a composition according any one of claims 1 to 14 in the manufacture of a medicament for use in the improved release profile of a drug with a rapidly changing solubility in the pH range 4.5 to 8.0.

19. A method of treatment of ulcerative colitis, Crohn's disease, irritable bowel syndrome and/or inflammatory bowel disease which method comprises administering a composition according to any one of claims 1 to 14 to a patient, preferably a human patient.

20. The use of a composition according any one of claims 1 to 14 in the manufacture of a medicament for the treatment of ulcerative colitis, Crohn's disease, irritable bowel syndrome and/or inflammatory bowel disease.
